(19) United States
(12) Patent Application Publication
(10) Pub. No.: US 2005/0037374 A1
(43) Pub. Date: Feb. 17, 2005

(54) Combined nanotechnology and sensor technologies for simultaneous diagnosis and treatment

(76) Inventors: Richard J. Melker, Gainesville, FL (US); Donn Michael Dennis, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/744,789

(22) Filed: Dec. 23, 2003

Related U.S. Application Data
(63) Continuation-in-part of application No. 10/345,532, filed on Jan. 16, 2003.
(63) Continuation-in-part of application No. 10/274,829, filed on Oct. 21, 2002.
(63) Continuation-in-part of application No. 10/154,201, filed on May 22, 2002.
(63) Continuation-in-part of application No. 09/708,789, filed on Nov. 8, 2000, now abandoned.
(60) Provisional application No. 60/292,962, filed on May 23, 2001.
(60) Provisional application No. 60/164,250, filed on Nov. 8, 1999.

(51) Int. Cl. C12Q 1/68; G01N 33/53

(52) U.S. Cl. 435/6; 435/7.1

(57) ABSTRACT

Systems and methods for diagnosing and/or treating conditions, diseases, or disorders. The present invention uses nanoparticle-based assemblies, which comprise a nanoparticle; a surrogate marker; and a means for detecting a specific chemical entity. Such nanoparticle-based assemblies combine nanotechnology and sensor technology to provide an efficient and accurate means for diagnosing a condition, disease, or disorder as well as for focused treatment regimens.

### CROSS-REFERENCE TO RELATED APPLICATION

This application is a continuation-in-part of co-pending U.S. patent application Ser. No. 10/345,532, filed Jan. 16, 2003; Ser. No. 10/274,829, filed Oct. 21, 2002; Ser. No. 10/154,201, filed May 22, 2002, which claims the benefit of U.S. Application Ser. No. 60/292,962, filed May 23, 2001; and Ser. No. 09/708,789, filed Nov. 8, 2000, which claims the benefit of U.S. Application Ser. No. 60/164,250, filed Nov. 8, 1999, all of which are hereby incorporated by reference herein in their entirety, including any figures, tables, or drawings.

### GOVERNMENT SUPPORT

The subject matter of this application has been supported by a research grant from the National Science Foundation (Grant Number NSF: EEC 02-10580). Accordingly, the government may have certain rights in this invention.

### BACKGROUND OF THE INVENTION

There is a great need for the development of efficient and accurate systems for the detection, notification, and treatment of a variety of medical conditions, disorders, and diseases. This requires an effective means for identifying in a patient the presence of specific chemical and/or biological agents including, but not limited to, nucleic acids, proteins, illicit drugs, toxins, pharmaceuticals, carcinogens, poisons, allergens, and infectious agents. Current methods of detecting such chemical or biological agents entail extraction of a sample into organic solvents, followed by analysis using stand-alone analytical systems such as gas-liquid chromatography and/or mass spectroscopy. These methods are time-consuming and often expensive. Moreover, these methods do not include simultaneous treatment of the condition, disorder, or disease associated with the chemical or biological agent in the patient.

Three recent advancements in medicine are particularly germane to expanding the potential of detecting chemical and/or biological agents, especially with regard to the treatment of disease: nanotechnology, biodetectors (biosensors), and the identification of biomarkers for specific diseases and/or conditions. Nanotechnology, such as nanoparticles, offers many advantages when used for applications such as the delivery of bioactive agents (i.e., DNA, AIDS drugs, gene therapy, immunosuppressants, chemotherapeutics), and drug uptake and degradation (i.e., enzyme encapsulation). For example, nanoparticles have been proposed as providing site-specific distribution of drugs to a target site. Appropriately sized particles have been proposed wherein such particles can be delivered to selected tissues to release their drug “payload” in a controlled and sustained manner.

The term “biodetectors” or “biosensors” relates to the use of naturally occurring and/or synthetic compounds as highly specific and extraordinarily sensitive detectors of various types of molecules and markers of disease. Naturally-occurring compounds such as antibodies have been used to provide molecular recognition for a wide variety of target molecules in diagnostic assays. Alternatively, synthetic compounds have been manufactured that mimic naturally-occurring mechanisms of DNA, RNA, and protein synthesis in cells to facilitate the detection of target chemical or biological agents.

Aptamers have recently been identified as potentially effective biosensors for molecules and compounds of scientific and commercial interest (see Brody, E. N. and L. Gold, “Aptamers as therapeutic and diagnostic agents,” J Biotechnol., 74(1):5-13 (2000) and Brody et al., “The use of aptamers in large arrays for molecular diagnostics,” Mol. Diagn., 4(4):381-8 (1999)). For example, aptamers have demonstrated greater specificity and robustness than antibody-based diagnostic technologies. In contrast to antibodies, whose identification and production completely rest on animals and/or cultured cells, both the identification and production of aptamers takes place in vitro without any requirement for animals or cells.

Aptamer synthesis is potentially far cheaper and reproducible than antibody-based diagnostic tests. Aptamers are produced by solid phase chemical synthesis, an accurate and reproducible process with consistency among production batches. An aptamer can be produced in large quantities by polymerase chain reaction (PCR) and once the sequence is known, can be assembled from individual naturally occurring nucleotides and/or synthetic nucleotides. Aptamers are stable to long-term storage at room temperature, and, if denatured, aptamers can easily be renatured, a feature not shared by antibodies. Furthermore, aptamers have the potential to measure concentrations of ligand in orders of magnitude lower (parts per trillion or even quadrillion) than those antibody-based diagnostic tests. These inherent characteristics of aptamers make them attractive for diagnostic applications.

A number of “molecular beacons” (often fluorescence compounds) can be attached to aptamers to provide a means for signaling the presence of and quantifying a target chemical or biological agent. For instance, an aptamer specific for cocaine has recently been synthesized (Stojanovic, M. N. et al., “Aptamer-based folding fluorescent sensor for cocaine,” J. Am. Chem. Soc., 123(21):4928:31 (2001)). A fluorescence beacon, which quenches when cocaine is reversibly bound to the aptamer is used with a photodetector to quantify the concentration of cocaine present. Aptamer-based biosensors can be used repeatedly, in contrast to antibody-based tests that can be used only once.

Of particular interest as a beacon are amplifying fluorescent polymers (AFP). AFPs with a high specificity to TNT and DNT have been developed. It has been noted that a detector based on AFP technology, with high specificity to TNT and DNT, can also detect propofol, an intravenous anesthetic agent, in extremely low concentration. The combination of AFP and aptamer technologies holds the promise of robust, reusable biosensors that can detect compounds in minute concentrations with high specificity.

The term “biomarker” refers to a biochemical in the body that has a particular molecular trait to make it useful for diagnosing a condition, disorder, or disease and for measuring or indicating the effects or progress of a condition, disorder, or disease. For example, common biomarkers found in a person's bodily fluids (i.e., breath or blood), and the respective diagnostic conditions of the person providing such biomarkers include, but are not limited to, acetaldehyde (source: ethanol; diagnosis: intoxication), acetone (source: acetoacetate; diagnosis: diet; ketogenic/diabetes), ammonia (source: deamination of amino acids; diagnosis: uremia and liver disease), CO (carbon monoxide) (source: CH₂Cl₂, elevated % COH; diagnosis: indoor air pollution), chloroform (source: halogenated compounds), dichlorobenzene (source: halogenated compounds), diethylamine (source: choline; diagnosis: intestinal bacterial overgrowth), H (hydrogen) (source: intestines; diagnosis: lactose intolerance), isoprene (source: fatty acid; diagnosis: metabolic stress), methanethiol (source: methionine; diagnosis: intestinal bacterial overgrowth), methylethylketone (source: fatty acid; diagnosis: indoor air pollution/diet), O-toluidine (source: carcinoma metabolite; diagnosis: bronchogenic carcinoma), pentane sulfides and sulfides (source: lipid peroxidation; diagnosis: myocardial infarction), H₂S (source: metabolism; diagnosis: periodontal disease/ovulation), MeS (source: metabolism; diagnosis: cirrhosis), and Me₂S (source: infection; diagnosis: trench mouth).

Medical science has also recognized the need to control, regulate and target the release of drugs in the body. Mechanisms of drug metabolism are extremely complex and are influenced by a number of factors including competitive binding on protein and red blood cells with other molecules; enzymatic activity, particularly in the liver; protein, and red blood cell concentration; and a myriad of other factors. Thus, the goals have been to provide: 1) less frequent drug administration, 2) constant and continuous therapeutic levels of a drug in the systemic circulation or at a specific target organ site, 3) a reduction in undesirable drug side effects, and 4) a reduction in the amount and dose concentration required to realize the desired therapeutic benefit.

During the past decade, a wide variety of drug delivery systems have been designed and evaluated which include, for example, 1) drug carriers based on proteins, polysaccharides, synthetic polymers, erythrocytes, DNA and liposomes, 2) microspheres containing an entrapped drug. In particular, serum albumin microspheres can be sustained and controlled by various stabilization procedures generally involving heat or chemical-crosslinking of the carrier matrix. However, very little technology is available that can detect and notify the user of a specific medical state in real-time as well as allow convenient, simultaneous treatment of the medical state. It is therefore desirable to develop a system that could accurately and efficiently detect/screen for target chemical and biological agents while simultaneously treating the corresponding condition, disorder, or disease, which would provide a significant cost and time benefit, expand medical practice, as well as improve patient quality of life.

### BRIEF SUMMARY OF THE INVENTION

The present invention provides nanostructures designed to release a marker (hereinafter the “surrogate marker”) in response to sensing a specific chemical entity (SCE) or a unique combination of SCEs in the body, which will, in turn, be readily detected in bodily fluids (i.e., exhaled breath, urine, etc.). The detection of a surrogate marker may in some cases be used to quantitatively relate the concentration of the SCE in the body. In other cases, detection of a surrogate marker can be used in a purely qualitative sense (to simply signal the presence of an SCE in the body without quantification).

The present invention provides systems and methods for notification/diagnosis of different physical conditions or disease/disorder states of a patient. This invention is based in part on nanostructure-based assemblies that include: a nanoparticle; a means for detecting an SCE; and a means for notifying the physician or healthcare provider that the SCE is present. In accordance with the present invention, compositions containing the nanostructure-based assemblies of the invention are administered to a patient for use in detecting and notifying in real time of a specific medical state.

In one embodiment, the nanostructure-based assemblies of the invention can be used to differentiate and signal types of blood cells and their concentrations in the patient. For example, levels of red blood cells (RBCs), white blood cells (WBCs), and platelets can be assessed using the systems and methods of the invention to diagnose and/or treat hematopoiesis abnormalities such as leukemia or assess changes in cellular contect (e.g., RBC content). Accordingly, the subject invention is useful in diagnosing and/or treating blood-based diseases or disorders including, without limitation, hemorrhagic diathesis (i.e., hemophilia, von Willebrand disease, Alexander's disease, Telfer's disease, Owren's parahemophilia, prothrombin deficiency); non-hemorrhagiparous coagulopathies (i.e., Fletcher factor deficiency, Flaujeac factor deficiency); thrombophilic coagulopathies (i.e., Ratnoff's disease, thrombomodulin deficiency); thrombocytopenia; anemias; and alterations in white blood cells (i.e., Pelger-Huët anomaly (PHA); Chediak-Higashi syndrome (CHS); Hegglin-May anomaly (HMA)).

In addition to providing notification/diagnosis, the systems and methods of the invention also enable substantially simultaneous treatment of a specific physical condition or disease/disorder state. In one embodiment, nanostructure-based assemblies include a means for detecting an SCE; a means for notifying the physician or healthcare provider that the SCE is present; and a means for treating the condition, disease, or disorder that is associated with the target SCE. Accordingly, the systems and methods of the present invention allow for substantially simultaneous diagnosis and treatment of the medical state.

In operation, a patient is administered a composition comprising a nanostructure-based assembly of the invention. The nanostructure-based assembly of the invention is composed of a nanoparticle that contains the following components: (a) a means for detecting an SCE; and (b) a surrogate marker. In another embodiment, the nanoparticle contains an additional component, (c) a “payload.” These components can be attached to any surface of the nanoparticle.

The SCE could be 1) attached to different types of cells (i.e., surface markers of diseased or normal cells), or 2) located in various bodily fluids (i.e., circulating markers of inflammatory disorders or cancer; therapeutic or illicit drugs) such as the blood. Thus, the SCE would include, but not be limited to, a biomarker or analyte such as a protein, DNA, RNA, oligonucleotides, sugars, nucleosides, nucleotides, aptamers or a variety of small therapeutic and/or illicit drug molecule targets.

An identification of an SCE by the SCE-detecting means affects the release of the surrogate marker from the nanoparticle. Because the surrogate marker is released from the nanoparticle only in the presence of an SCE, detection of the surrogate marker provides notice that the SCE is present in the patient and consequently, allows diagnosis of the specific condition, disorder, or disease associated with the SCE.

Further, the detection of an SCE can also cause the substantially simultaneous release of a payload, when provided, with the surrogate marker. The payload is designed to prevent, alleviate, and/or cure the specific condition, disorder, or disease associated with the SCE. Thus, with concentrated delivery of the payload agent at the desired organ or tissue site, specific therapeutic effects can now be realized with minimized side effects, thereby permitting enhanced desired therapeutic activity and the use of decreased dosage amounts. Thus, the detection of the surrogate marker would also serve as an indication that the payload has been released.

The present invention can be used to diagnose, notify, and track the progress of therapeutic interventions for a wide variety of disease states in a convenient non-invasive manner using a point-of-care (POC) approach, either in a patient's home or in a health care provider area.

The present invention provides novel systems and methods for improving the quality of health care by enabling the following benefits in a non-invasive manner: 1) allow early detection of disease and identify those at risk of developing the disease, 2) provide an indication of the prognosis of the disease, 3) allow for accurate monitoring of therapeutic efficacy and drug compliance, 4) allow for detection of disease recurrence; and 5) allow for focused treatment of the disease, disorder, or condition.

In accordance with the present invention, the SCE-detecting means includes well-known biodetectors or biosensors. Such biodetectors or biosensors include naturally occurring and/or synthetic compounds having high specificity and sensitivity to chemical and/or biological compounds of interest. Suitable biodetectors or biosensors of the invention include, but are not limited to, antibodies, proteins, and aptamers.

In one embodiment, the detecting means has the capability of localizing the nanostructure-based assembly to the vicinity of the SCE. In other embodiments, the detecting means also has the capability of cellular localization (i.e., delivering the nanostructure-based assembly to a cancer cell) or subcellular localization (i.e., delivering the nanostructure-based assembly to a nucleus within a cancer cell).

According to the present invention, the surrogate marker is an innocuous compound that is readily detectable in bodily fluid samples. In preferred embodiments, the surrogate marker is a volatile compound (e.g., dimethyl sulfoxide—DMSO).

The “payload,” as contemplated herein, is a therapeutic bioactive agent used in the prevention, cure, or alleviation of a medical condition, disorder, or disease.

In one embodiment, the nanoparticle-based assemblies of the invention are composed of biodegradable substances. In another embodiment, the nanoparticle-based assemblies are composed of biocompatible substances.

In another embodiment of the present invention, the nanoparticle of the nanostructure-based assembly has a hollow body defining an inner void, which contains the surrogate marker and payload. Release of the surrogate marker and payload is controlled by an end-cap to which a means for detecting an SCE is attached. The detecting means is designed to undergo a conformational change upon detecting the SCE to detach the end-cap from the nanoparticle and release both the surrogate marker and the payload. In certain embodiments, the nanoparticle contains only the surrogate marker.

In a related embodiment, the detecting means is attached to the outer surface of the nanoparticle. The controlled release of the surrogate marker and, when present, payload is accomplished by the release of the end-cap, which is attached to the nanoparticle via chemically labile bonds.

Yet another embodiment provides a nanoparticle that has the detecting means, the surrogate marker, and the payload (when present) applied to the outside of the surface of the nanoparticle. All of these components are attached to the surface of the nanoparticle via chemically labile bonds, which allow for the release of these components under specific conditions.

After administration of the nanostructure-based assembly to a patient, a sample of bodily fluid is collected from the patient for analysis. According to the invention, a sample of bodily fluid includes, but is not limited to, exhaled breath (including cough, sneeze), blood, urine, sweat, mucous, semen, bile, feces, saliva, lymph fluid, blood plasma, amniotic fluid, glandular fluid, sputum, and cerebral spinal fluid. The bodily fluid sample is analyzed for the presence of the surrogate marker, which indicates the presence of the SCE in the patient and consequently, allows for the diagnosis of the condition, disease, or disorder associated with the SCE.

For analysis of bodily fluid samples to detect the presence of the surrogate marker, sensor technology is applied in accordance with the present invention. Contemplated sensor technology includes, but is not limited to, previously disclosed sensor technology such as semiconductor gas sensor technology, conductive polymer gas sensor technology, surface acoustic wave gas sensor technology, and immunoassays.

### BRIEF DESCRIPTION OF THE FIGURES

FIG. 1 is a table illustrating certain specific chemical compounds that can be detected using the nanoparticle-based assemblies of the present invention.

### DETAILED DESCRIPTION OF THE INVENTION

The present invention is directed to the efficient, accurate, and real-time identification, notification, and/or treatment of a condition, disease or disorder. The systems and methods of the invention utilize nanostructure-based assemblies that contain a nanoparticle, a means for detecting a target SCE, and a surrogate marker. In certain embodiments, nanostructure-based assembles also include a payload to provide localized treatment of the condition, disease, or disorder. Commonly available sensor technology is used by the present invention to detect the presence of a surrogate marker released from a nanostructure-based assembly in a bodily fluid sample.

In operation, after administration of the nanostructure-based assemblies of the invention to a patient, a bodily fluid sample is collected from the patient, to which sensor technology is applied to detect the presence of surrogate markers. Surrogate markers (and when provided, payload) are generally released into the patient when nanostructure-based assemblies are in the presence of target SCEs. Specifically, bioactive interaction between the SCE-detector and the target SCE induces the release of the surrogate marker and payload from the nanoparticle. Advantageously, the concentration of the released surrogate marker is proportional to the amount of SCE present in the bodily fluid sample, which can be measured using quantitative sensor technology known in the art.

Definitions

Unless otherwise stated, the following terms used in the specification and claims have the meanings given below.

The term “aptamer,” as used herein, refers to a non-naturally occurring oligonucleotide chain that has a specific action on an SCE of interest. A specific action includes, but is not limited to, binding of the target SCE, catalytically changing the target SCE, and reacting with the target SCE in a way which modifies/alters the SCE or the functional activity of the SCE. The aptamers of the invention preferably specifically bind to a target SCE and/or react with the target SCE in a way which modifies/alters the SCE or the functional activity of the SCE.

Aptamers include nucleic acids that are identified from a candidate mixture of nucleic acids. In a preferred embodiment, aptamers include nucleic acid sequences that are substantially homologous to the nucleic acid ligands isolated by the SELEX method. Substantially homologous is meant a degree of primary sequence homology in excess of 70%, most preferably in excess of 80%.

The “SELEX™” methodology, as used herein, involves the combination of selected nucleic acid ligands, which interact with a target SCE in a desired action, for example binding to a protein, with amplification of those selected nucleic acids. Optional iterative cycling of the selection/amplification steps allows selection of one or a small number of nucleic acids, which interact most strongly with the target SCE from a pool, which contains a very large number of nucleic acids. Cycling of the selection/amplification procedure is continued until a selected goal is achieved. The SELEX methodology is described in the following U.S. patents and patent applications: U.S. patent application Ser. No. 07/536,428 and U.S. Pat. Nos. 5,475,096 and 5,270,163.

The term “indicator aptamers,” as used herein, refers to aptamers to which molecular beacons are attached, such as those described in U.S. Pat. Nos. 6,399,302 and 5,989,823.

The term “molecular beacons,” as used herein, refers to a molecule or group of molecules (i.e., a nucleic acid molecule hybridized to an energy transfer complex or chromophore(s)) that can become detectable and can be attached to a biodetector/biosensor under preselected conditions. For example, an embodiment of the present invention includes an aptamer-bound fluorescence beacon that (a) quenches when a target SCE is reversibly bound to the aptamer and (b) is detectable with a photodetector to quantify the concentration of target SCE present.

As used herein, the term “specific chemical entity” or “SCE,” refers to naturally occurring and/or synthetic compounds, which are a marker of a condition (i.e., drug abuse), disease state (i.e., infectious diseases), disorder (i.e., neurological disorders), or a normal or pathologic process that occurs in a patient (i.e., drug metabolism). The term SCE can also refer to, without limitation, any substance, including an analyte, biomarker, and chemical and/or biological agents that can be measured in an analytical procedure.

SCEs that are detected by the present invention include, but are not limited to, the following metabolites or compounds commonly found in bodily fluids: acetaldehyde (source: ethanol; diagnosis: intoxication), acetone (source: acetoacetate; diagnosis: diet or ketogenic/diabetes), ammonia (source: deamination of amino acids; diagnosis: uremia and liver disease), CO (carbon monoxide) (source: CH₂Cl₂, elevated % COHb; diagnosis: indoor air pollution), chloroform (source: halogenated compounds), dichlorobenzene (source: halogenated compounds), diethylamine (source: choline; diagnosis: intestinal bacterial overgrowth), H (hydrogen) (source: intestines; diagnosis: lactose intolerance), isoprene (source: fatty acid; diagnosis: metabolic stress), methanethiol (source: methionine; diagnosis: intestinal bacterial overgrowth), methylethylketone (source: fatty acid; diagnosis: indoor air pollution/diet), O-toluidine (source: carcinoma metabolite; diagnosis: bronchogenic carcinoma), pentane sulfides and sulfides (source: lipid peroxidation; diagnosis: myocardial infarction), H₂S (source: metabolism; diagnosis: periodontal disease/ovulation), MeS (source: metabolism; diagnosis: cirrhosis), Me₂S (source: infection; diagnosis: trench mouth), αII-spectrin breakdown products and/or isoprostanes (source: cerebral spinal fluid, blood; diagnosis: traumatic or other brain injuries); prostate specific antigen (source: prostate cells; diagnosis: prostate cancer); and GLXA (source: gylcolipid in Chlamydia; diagnosis: Chlamydia).

Additional SCEs detected by the present invention include, but are not limited to, any nucleotide sequences provided in a genomic or cDNA library; any peptides in a phage displayed library; illicit, illegal, and/or controlled substances including drugs of abuse (i.e., amphetamines, analgesics, barbiturates, club drugs, cocaine, crack cocaine, depressants, designer drugs, ecstasy, Gamma Hydroxy Butyrate—GHB, hallucinogens, heroin/morphine, inhalants, ketamine, lysergic acid diethylamide—LSD, marijuana, methamphetamines, opiates/narcotics, phencyclidine—PCP, prescription drugs, psychedelics, Rohypnol, steroids, and stimulants); allergens (i.e., pollen, spores, dander, peanuts, eggs, and shellfish); toxins (i.e., mercury, lead, other heavy metals, and Clostridium Difficile toxin); carcinogens (i.e., acetaldehyde, beryllium compounds, chromium, dichlorodiphenyltrichloroethane (DDT), estrogens, N-methyl-N′-nitro-N-nitrosoguanidine (MNNG), and radon); infectious agents (i.e., Bordettella bronchiseptica, citrobacter, Escherichia coli, hepatitis viruses, herpes, immunodeficiency viruses, influenza virus, Listeria, micrococcus, mycobacterium, rabies virus, rhinovirus, rubella virus, Salmonella, and yellow fever virus), cell markers for diseases (i.e., T cell markers, B cell markers, myeloid/monocytic markers, maturity status markers for Leukemia, analplastic lymphoma, Hodgkins' disease; α-Fetoprotein (AFP) as an indicator of hepatocellular carcinoma and non-seminomatous testicular cancer; β2-Microglobulin (b2-M) as an indicator of active disease, cell turnover, tumor presence, and inflammatory diseases; and Beta Human Chorionic Gonadotropin (b HCG)) is a tumor marker for gestational trophoblastic diseases and germ cell tumors of the ovary or testis).

The term “bodily fluid,” as used herein, refers to a mixture of molecules obtained from a patient. Bodily fluids include, but are not limited to, exhaled breath, whole blood, blood plasma, urine, semen, saliva, lymph fluid, meningal fluid, amniotic fluid, glandular fluid, sputum, feces, sweat, mucous, and cerebrospinal fluid. Bodily fluid also includes experimentally separated fractions of all of the preceding solutions or mixtures containing homogenized solid material, such as feces, tissues, and biopsy samples.

The term “SCE-detector” or “SCE-detecting means,” as used herein, refers to the use of biodetectors and/or biosensors, including naturally-occurring and/or synthetic compounds, as highly specific and sensitive detectors of various types of SCEs. Naturally-occurring compounds such as antibodies, proteins, receptor ligands, and receptor proteins have been used to provide molecular recognition for a wide variety of target molecules in diagnostic assays. Alternatively, synthetic compounds such as aptamers have been manufactured that mimic naturally occurring mechanisms of DNA, RNA, and protein synthesis in cells to facilitate detection of target SCEs.

The term “surrogate marker,” as used herein, refers to a molecule or compound that is innocuous to the patient and detectable by means of its physical or chemical properties. As such, surrogate markers are detectable by a number of sensor technologies known in the art including, but not limited to, flow cytometers, semiconductive gas sensors; mass spectrometers; infrared (IR), ultraviolet (UV), visible, or fluorescence spectrophotometers; gas chromatography, conductive polymer gas sensor technology; surface acoustic wave gas sensor technology; immunoassay technology, and amplifying fluorescent polymer (AFP) sensor technology. The surrogate markers of the invention include federally approved products categorized as GRAS (“generally recognized as safe”) as well as other compounds not formally designated as GRAS which have suitable toxicological and physicochemical properties to be detected in accordance with the systems and methods of the subject invention. In preferred embodiments, the surrogate marker is a volatile marker detectable in bodily fluids, in particular blood and breath.

A “patient,” as used herein, describes an organism, including mammals, to which treatment with the compositions according to the present invention is provided. Mammalian species that benefit from the disclosed methods of treatment include, and are not limited to, apes, chimpanzees, orangutans, humans, monkeys; and domesticated animals (e.g., pets) such as dogs, cats, mice, rats, guinea pigs, and hamsters.

As used herein, the term “pharmaceutically acceptable carrier” means a carrier that is useful in preparing a pharmaceutical composition that is generally compatible with the other ingredients of the composition, not deleterious to the patient, and neither biologically nor otherwise undesirable, and includes a carrier that is acceptable for veterinary use as well as human pharmaceutical use. “A pharmaceutically acceptable carrier” as used in the specification and claims includes both one and more than one such carrier.

As used herein, a “biodegradable” substance refers to a substance that can be decomposed by biological agents or by natural activity within an organism. Examples of contemplated biodegradable polymers include, but are not limited to: polyesters such as poly(caprolactone), poly(glycolic acid), poly(lactic acid), and polyhydroxybutrate; polyanhydrides such as poly(adipic anhydride) and poly(maleic anhydride); polydioxanone; polyamines; polyamides; polyurethanes; polyesteramides; polyorthoesters; polyacetals; polyketals; polycarbonates; polyorthocarbonates; polyphosphazenes; poly(malic acid); poly(amino acids); polyvinylpyrrolidone; poly(methyl vinyl ether); poly(alkylene oxalate); poly(alkylene succinate); polyhydroxycellulose; chitin; chitosan; and copolymers and mixtures thereof.

As used herein, a “biocompatible” substance includes those substances that are compatible with and have demonstrated no significant toxic effects on living organisms. Examples of contemplated biocompatible polymers include PLG (Poly(lactide-co-glycolide)), poly(ethylene glycol), and copolymers of poly(ethylene oxide) with poly(L-Lactic acid) or with poly(β-benzyl-L-aspartate). In a preferred embodiment, biocompatibility includes immunogenic compatability. An immunogenically compatible substance can include a substance that, when introduced into a body, does not significantly elicit humoral or cell-based immunity.

As used herein, “treating” or “treatment” includes: (1) preventing the condition, disorder, or disease (i.e., inhibiting the development of clinical symptoms of a disease in a mammal that may be exposed to or predisposed to the disease but does not yet experience or display symptoms of the disease); (2) inhibiting the condition, disorder, or disease (i.e., arresting the development of the condition or its clinical symptoms), or (3) relieving the condition, disorder, or disease (i.e., causing regression of the condition/disorder/disease or its clinical symptoms).

The term, “payload” or “payload material,” as used herein, refers to bioactive agents for treatment.

The term “therapeutically effective amount,” as used herein, means the amount of a compound that, when administered to a mammal for treating a medical state, is sufficient to effect such treatment for the medical state. The “therapeutically effective amount” will vary depending on the medication, the condition/disorder/disease state being treated, the severity of the condition/disorder/disease treated, the age and relative health of the patient, the route and form of administration, the judgment of the attending medical practitioner, and other factors.

Nanoparticles

Nanostructure-based assemblies offer timely, and effective detection, notification, and treatment of a condition, disorder, or disease. Such assemblies are based on nanoparticles, which provide a mechanism for the targeted delivery and release of detectable markers and/or bioactive treatment agents to selected sites within the body.

According to the present invention, nanoparticles can be produced in a wide range of sizes and shapes, and composed of a wide range of materials, or combination of materials, optimized for in-vivo administration. Contemplated shapes include, but are not limited to, spherical, elliptical, cubic, cylindrical, tetrahedron, polyhedral, irregular-prismatic, icosahedral, and cubo-octahedral forms. Nanoparticles intended for in-vivo use are of any dimension, preferably with a maximum dimension less than 500 nm, so as to ensure proper distribution at the microvasculatoure level, without any occlusion of blood flow. More preferably, the nanoparticles of the subject invention are of a dimension less than 100-150 nm. The “maximum dimension” of a nanoparticles is the maximum distance between any two points in the nanoparticle. In a preferred embodiment, the nanoparticles are in the form of tubular bodies (also known as “nanotubes”), which are either hollow or solid and include either open ends or one or both closed ends.

Methods of preparation of nanoparticles are well known in the art. For example, the preparation of monodisperse sol-gel silica nanospheres using the well-known Stober process is described in Vacassy, R. et al., “Synthesis of Microporous Silica Spheres,” J. Colloids and Interface Science, 227, 302 (2000).

Nanoparticles, in accordance with the present invention, can be prepared from a single material or a combination of materials. For example, nanotubes can be prepared from either one or a combination of materials including, but not limited to, polymers, semiconductors, carbons, or Li⁺ intercalation materials. Metal nanoparticles include those made from gold or silver. Semi-conductor nanoparticles include those made from silicon or germanium. Polymer nanoparticles include those made from biocompatible or biodegradable polymers. The ability to make nanoparticles from a wide variety of materials or combination of materials allows the creation of nanoparticles with desired biochemical properties such as biocompatibility, including immunogenic compatibility, and/or, biodegradability. In comparison, certain biological delivery systems, such as viral vectors, can cause significant immunogenic phenomena.

Nanoparticles of the present invention can be synthesized using a template synthesis method. For example, nanoparticles can be synthesized using templates prepared from glass (Tonucci, R. J. et al., Science 258, 783 (1992)), xeolite (Beck, J. S. et al., J. Am. Chem. Soc., 114, 10834 (1992)), and a variety of other materials (Ozin, G. A., Adv. Mater., 4, 612 1992)). Alternatively, nanoparticles can be prepared using a self-assembly process, as described in Wang, Z. L., “Structural Analysis of Self-Assembling Nanocrystal Superlattices,” Adv. Mater., 10(1): 13-30 (1998).

In one embodiment, a nanostructure-based assembly of the invention contains a nanoparticle, which has one or more surfaces functionalized to allow attachment of SCE-detectors to the surface. Such “functionalized” nanoparticles have at least one surface modified to allow for directed (also referred to as “vectoring”) delivery and/or controlled release of the payload and surrogate marker. In certain embodiments, the nanoparticle is formed with an interior void. Different chemical and/or biochemical functional groups can be applied to the inside and/or outside surfaces of the nanoparticle to enable the attachment of an SCE-detector, surrogate marker, and/or payload on a nanoparticle surface.

In another embodiment, the nanostructure-based assembly contains a nanoparticle formed with an interior void to contain a surrogate marker, a payload, and a detachable end-cap with an SCE-detector attached thereto. In the presence of a target SCE, the SCE-detector mechanically detaches the end-cap from the nanoparticle to release the surrogate marker for analysis by sensor technology. Simultaneously, the payload is released for the treatment of a condition, disorder, or disease.

In a preferred embodiment, the nanoparticle is in the form of a nanotube that is hollow and has a first open end and a second closed end. A surrogate marker and payload are enclosed within the hollow interior of the nanotube. The first open end is blocked with an aptamer-bound end-cap that prevents the release of the surrogate marker and payload located within the hollow interior of the nanotube.

Upon detecting a target SCE by the aptamer attached to the end-cap, the surrogate marker and payload are released with the uncapping of the nanoparticle. The uncapping mechanism may require the use of energy-bearing biomolecular motors such as, but not limited to, the actin-based system (Dickinson, R. B. and D. L. Purich, “Clamped filament elongation model for actin-based motors,” Biophys J, 82:605-617 (2002)). Once the nanoparticle is uncapped, the released surrogate marker can then be detected using sensor technology known in the art including, but not limited to, gas chromatography, electronic noses, spectrophotometers to detect the surrogate marker's infrared (IF), ultraviolet (UV), or visible absorbance or fluorescence, or mass spectrometers. Further, the release of the payload ensures localized release of treatment at the desired organ or tissue site, thereby permitting enhanced, desired therapeutic activity and decreased use of dosage amounts.

Nanotubes

A number of patents and publications describe nanoparticles in the form of tubes (nanotubes). For example, U.S. Pat. No. 5,482,601 to Ohshima et al. describes a method for producing carbon nanotubes. Other methods for making and using nanotubes include the non-carbon nanotubes of Zettl et al., U.S. Pat. No. 6,063,243, and the functionalized nanotubes of Fisher et al., U.S. Pat. No. 6,203,814.

For nanotubes, synthesis occurs within the membrane pores of a microporous membrane or other solid, as described in Charles R. Martin, “Nanomaterials: A Membrane-Based Synthetic Approach,” Science, 266:1961-1966 (1994), using electrochemical or chemical methods. Depending on the membrane and synthetic method used, the nanotubes may be solid or hollow. Template membrane pore diameters can be varied to produce nanotubes having diameters as small as 5 nm to as large as 100 μm. Likewise, the template membrane thickness can be varied to give nanotubes having a length from as small as 5 nm to as large as 100 μm. Preferably, when the nanotube is intended for in vivo use, the nanotube is of length less than 500 μm and diameter less than 200 nm. Especially preferred nanotubes for in vivo use have a maximum dimension less than 100 nm.

“Track-etch” polymeric or porous alumina membranes can be used in the preparation of nanotubes. Track-etch membranes prepared from polycarbonate and polyester are available from suppliers such as Osmonics (Minnetonka, Minn.) and Whatman (Maidstone, Kent UK). Track-etch membranes contain randomly distributed cylindrical pores of uniform diameter that run through the entire thickness of the membrane. Pore diameters as small as 10 nm are commercially available at pore densities of up to 10⁹ pores per square centimeter.

Porous alumina membranes, which are commercially available from Whatman (Maidstone, Kent UK), are prepared electronically from aluminum metal. Pore diameters as small as 5 nm can be achieved at pore densitites as high as 10¹¹ pores per square centimeter. Membranes can be prepared having the membrane thickness from as small as 100 nm to as large as 100 μm.

Nanotubes can be synthesized such that both ends of the nanotube are open. Alternatively, nanotubes having one open end can be synthesized. Solid nanotubes can also be synthesized.

Nanotubes with one closed end can be produced by template synthesis, as described above. For example, nanotubes having one closed end can be prepared by terminating the pores in the alumina template into a non-porous alumina barrier layer prior to removal of the alumina template membrane from the substrate aluminum surface (Hornyak, G. L., et al., “Fabrication, Characterization and Optical Properties of Gold-Nanoparticle/Porous-Alumina Composites: The Non-Scattering Maxwell-Garnett Limit,” J. Phys. Chem. B., 101:1548-1555 (1997)). Specifically, the non-porous alumina barrier layer is removed when the alumina membrane is stripped off of the aluminum surface. However, if the template synthesis is completed before removal of the alumina from the aluminum, the bottoms of the nanotubes are closed. Dissolution of the alumina then liberates the nanotubes that are closed at one end and open at the other end.

Suitable end-caps used to block a nanotube opening include, for example, nanoparticles having a diameter slightly larger than the inside diameter of the nanoparticle so as to occlude the open end of the nanoparticle. End-caps are any piece of matter and can be composed of materials that are chemically or physically similar (or dissimilar) to the nanoparticle. The end-cap can be a particle that has a maximum dimension of less than 100 μm. In a preferred embodiment, the end-cap is of a spherical or spheroidal form. However, end-caps of other shapes, including ellipsoidal, cylindrical, and irregular, can also be used.

A suitable end-cap can be attached to a nanotube by covalent bonds. For example, silica nanotubes and particles can be linked by disulphide bonds. Initially, the surface at the ends of silica nanotubes is functionalized with a —SH linker. This can be performed while the nanotubes are still embedded in the pores of the template membrane. This allows activation of the end surface without changing the chemical properties of the outer surface of the nanotubes.

If necessary, the inner surfaces of the nanotubes are protected with, for example, a silane group such as (Me—O)₃—(CH₂)₃—OH. After the protection step, the silica surface layers at the nanotube mouths are removed to expose fresh silica. The freshly-exposed silica will be reacted with the silane, such as (Me—O)₃—Si—(CH₂)₃—SH to attach the requisite —SH linker to the mouths of the nanotubes. The length of the alkyl chain in this silane can be varied to allow placement of the —SH linker any desired distance from the nanotube mouth. These —SH functionalities are then reacted with pyridine disulfide in order to obtain nanotubes with an activated disulfide bond at the nanotube ends.

The surface of the end-cap is then functionalized with the same —SH containing silane used on the mouths of the nanotubes. Hence, nanotubes with an activated disulfide at their mouths and end-caps with an —SH group on their surface are available for linkage through disulfide bond formation.

Other types of covalent bonds, for example amide and ester bonds, can be used to attach an end-cap to the nanotube. Siloxane based linking can also be used. This would be particularly useful when the cap is composed of silica as the silanol sites on the silica surface reacts spontaneously with siloxanes to form a covalent oxygen-silicon bond. For metal based nanotubes or end-caps, thiol linkers can be used for attachment. For example, molecule (Me—O)₃—Si—(CH₂)₃—SH could be attached to a silica nanotube and a gold nanoparticle attached as the end-cap using the —SH end of this molecule. It is well known that such thiols form spontaneous As—S bonds with gold surfaces.

Contemplated end-caps for the invention include nanoparticles that can be electrophoretically placed within the mouths of nanotubes so that the entire mouth of the nanotube is blocked when disulfide bonds are formed between the nanotube and the nanoparticle as described in Miller, S. A. and C. R. Martin, “Electroosmotic Flow in Carbon Nanotube Membranes,” J. Am. Chem. Soc., 123(49):12335-12342 (2001).

For example, a nanotube containing membrane is mounted in a U-tube cell with Platinum electrodes immersed into the buffer solution on either side of the membrane. The —SH-functionalized end-caps are added to the cathode half-cell. The buffer solution is maintained at pH=7 so that a small fraction of the —SH groups on the end-caps are deprotonated. These negatively charged particles are driven into the mouths of the nanotubes electrophoretically by using the Platinum electrodes to pass a constant current through the membrane. Hence, the electrophoretic force causes the end-caps to nestle into the nanotube mouths, where disulfide bond formation will occur.

As an alternative to the electrophoretic assembly method, —SH labeled end-caps can be suspended in solution together with the activated disulfide labeled nanotubes. Here, the nanoparticle caps can spontaneously self-assemble to the nanotubes. The self-assembly of gold nanospheres and latex particles to template prepared polymeric and metal nanowires is described by Sapp, S. A. et al., “Using Template-Synthesized Micro- and Nanowires as Building Blocks for Self-Assembly of Supramolecular Architectures,” Chem. Mater., 11:1183-1185 (1999).

In addition to —SH linking, other covalent linking methods can be used to link nanotubes and end-caps. Non-covalent linking methods can be used. These include, for example, DNA hybridization (Mirkin, C. A., “Programming the Self-Assembly of Two and Three-Dimensional Architectures with DNA and Nanoscale Inorganic Building Blocks,” Inorg. Chem., 39:2258-2272 (2000)), the biotin/avidin interaction (Connolly, S. and D. Fitzmaurice, “Programmed Assembly of Gold Nanocrystals in Aqueous Solution,” Adv. Mater., 11:1202-1205 (1999)), and antigen/antibody interactions (Shenton, W. et al., “Directed Self-Assembly of Nanoparticles into Macroscopic Materials Using Antibody-Antigen Recognition,” Adv. Mater., 11:449 (1999)).

Preferred nanotubes are those comprising silica or polymers. Silica nanotubes can be prepared using sol-gel template synthesis, as described in Lakshmi, B. B. et al., “Sol-Gel Template Synthesis of Semiconductor Oxide Micro- and Nanostructures,” Chem. Mater., 9:2544-2550 (1997); Lakshmi, B. B. et al., “Sol-Gel Template Synthesis of Semiconductor Nanostructures,” Chem Mater., 9:857-862 (1997). The template membrane is immersed into a standard tetraethylorthosilicate sol so that the sol fills the pores. After the desired emersion time, the membrane is removed, dried in air, and then cured at 150° C. This yields silica nanotubes lining the pore walls of the membrane plus silica surface films on both faces of the membrane. The surface films are removed by briefly polishing with slurry of alumina particles. The nanotubes are then liberated by dissolving the template membrane and collected by filtration.

The outside diameter of the nanotube can be controlled by varying the pore diameter of the template membrane, the length of the nanotube can be controlled by varying the thickness of the template membranes, and the inside diameter of the nanotube can be controlled by varying the immersion time in the sol.

Polymer nanotubes can be prepared from many substances that are composed of monomer units. “Monomer units,” as used herein, refers to the individual moieties that are repeated to form “polymers.” Multiple monomer units are covalently attached when tin the form of a backbone of a polymer. Polymers that are made from at least two different types of monomer units are referred to as “copolymers.” Polymerizing or copolymerizing describes the process by which multiple monomers are reacted to form covalently linked monomer units that form polymers or copolymers, respectively. A discussion of polymers, monomer units, and the monomers from which they are made may be found in Stevens, Polymer Chemistry: An Invitation, 3^{rd} ed., Oxford University Press (1999).

Polymeric nanotubes can be prepared using a solution deposition method as described in Depak, V. M. and C. R. Martin, “Preparation of Polymeric Micro- and Nanostructures Using a Template-Based Deposition Method,” Chem. Mater., 11:1363-1367 (1999). This method entails depositing a solution of the desired polymer within the pores of the template membrane and allowing the solvent to evaporate. In addition, polymer nanotubes can be prepared by polymerizing a monomer of a monomer within the pore as described by Martin, C. R., “Template Synthesis of Electronically Conductive Polymer Nanostructures,” Acc. Chem. Res., 28:61-68 (1995).

Preferred polymers include polystyrene, polyorganosiloxane, poly(methyl methacrylate), polystyrene, polylactic acids, and other biodegradable polymers, acrylic latexes, polyorganosiloxane, cellulose, polyethylene, poly(vinyl chloride), poly(ethyl methacrylate), poly(tetrafluoroethylene), poly(4-iodostyrene/divinylbenzene), poly(4-vinylpyridine/divinylbenzene), poly(styrene/divinyl benzene), crosslinked melamine particles, phenolic polymer colloids, polyamide 6/6, natural rubber, naturally occurring biopolymers such as algenates, and collagen, or mixtures thereof.

When the nanotubes are to be introduced into a patient, for example, when used as a nanostructure-based assembly for the detection, notification, and treatment of a disease, biodegradable polymers and biocompatible polymers are especially preferred. A “biodegradable” substance is a substance that can be broken down by the action of living organisms. Examples of useful biodegradable polymers include polyesters, such as poly(caprolactone), poly(glycolic acid), poly(lactic acid), and poly(hydroxybutryate); polyanhydrides, such as poly(adipic anhydride) and poly(maleic anhydride); polydioxanone; polyamines; polyamides; polyurethanes; polyesteramides; polyorthoesters; polyacetals; polyketals; polycarbonates; polyorthocarbonates; polyphosphazenes; poly(malic acid); poly(amino acids); polyvinylpyrrolidone; poly(methyl vinyl ether); poly(alkylene oxalate); poly(alkylene succinate); polyhydroxycellulose; chitin; chitosan; and copolymers and mixtures thereof.

“Biocompatible” substances are substances that are compatible with and have no significant toxic effect on living organisms. Preferably, biocompatibility includes immunogenic compatibility. An “immunogenically compatible” substance is a substance that, when introduced into a body, does not significantly elicit humoral or cell-based immunity. Examples of biocompatible polymers include PLG [Poly(lactide-co-glycolide)], poly(ethylene glycol), copolymers of poly(ethylene oxide) with poly(L-Lactic acid) or with poly(β-benzyl-L-aspartate. In addition, a number of approaches can be used to make a nanotube surface biocompatible and “stealthy.” For example, this can be accomplished by attaching a PEG-maleimide to the chain-end thiols on the outer surfaces of the nanotube. If the nanotube is composed of Au or similar metals, the PEG chain can be attached by a thiol linker as described in Yu, S.; Lee, S. B.: Kang, M.: Martin, C. R. “Size-Based Protein Separations in Poly(ethylene glycol)-Derivatized Gold Nanotubule Membranes,” Nano Letters, 1:495-498 (2001). Other examples of biocompatible polymers and surface treatments can be found in Majeti N. V. Ravi Kumar, “Nano and Microparticles as Controlled Drug Delivery Devices” J. Pharm. Pharmaceut. Sci. 3(2): 234-258 (2000), the contents of which are incorporated by this reference.

In one embodiment of the invention, a nanostructure-based assembly includes a nanotube with a hollow interior comprising a surrogate marker and/or payload material. The nanotube is constructed using known methods such as those disclosed in U.S. patent application Ser. No. 10/274,829, filed Oct. 21, 2002. The nanotube further includes a detecting means for localizing the nanostructure-based assembly to a target SCE. The surrogate marker and payload material are released from the nanostructure-based assembly when in the presence of a target SCE.

In a related embodiment, release of the surrogate marker and/or payload material in the hollow void is achieved by “uncapping” the nanotube. An end-cap is placed over an opening to the void to function as a means for controlling the release of the contents therein (i.e., surrogate marker and/or payload material). Methods for attaching an end-cap to a nanoparticle include, but are not limited to, using: electrostatic attraction, hydrogen bonding, acid and/or basic sites located on the end-cap/nanoparticle, covalent bonds, and other chemical linkages.

In a preferred embodiment, the detecting means is attached to the end-cap to affect the release of the surrogate marker and/or payload material via uncapping of the nanoparticle. For example, the uncapping mechanism is based upon the detection by the detecting means of certain SCEs including for example, surface markers on cell types (i.e., cancer cells), proteins in the blood (i.e., PSA for prostate cancer), or drugs in the body (i.e., illicit drugs or therapeutic drugs). The uncapping mechanism may require the use of energy-bearing biomolecular motors such as, but not limited to, the actin-based system (Dickinson, R. B. and D. L. Purich, “Clamped filament elongation model for actin-based motors,” Biophys J, 82:605-617 (2002)).

The released surrogate marker can then be detected using sensor technology known in the art including, but not limited to, gas chromatography, electronic noses, spectrophotometers to detect the detectable biomarker's infrared (IF), ultraviolet (UV), or visible absorbance or fluorescence, or mass spectrometers.

Functionalization of the Nanoparticles

According to the present invention, nanoparticles can be prepared having different chemically or biochemically functionalized surfaces to enable attachment of an SCE-detecting means, surrogate marker, and/or payload. Methods used to functionalize a nanoparticle surface depend on the composition of the nanoparticle and are well known in the art. For example, functionalization of silica nanoparticles is accomplished using silane chemistry. With silane chemistry, different functional groups can be attached to the surfaces of the nanoparticle by attaching a functional group to the nanoparticle surface while the nanoparticles are embedded within the pores of the template. Then, a hydrolytically unstable silane is reacted with the surface silanol sites on the nanoparticle to obtain covalent oxygen/silicon bonds between the surface and the silane. Additional functional groups can also be attached to the nanoparticle surface after dissolution of the template.

The surface of polymer nanoparticles can also be functionalized using well known chemical methods. For example, methods employed for polylactide synthesis allow for differential end-functionalization. Polymerization occurs by an insertion mechanism mediated by Lewis acids such as Sn²⁺ whose bonds with oxygen have significant covalent character. An alcohol complexed with the metal ion initiates polymerization, which continues by stepwise ring-opening of the lactide monomers to generate a new alkoxide-metal complex capable of chain growth. The polymer molecular weight can be controlled by the molar ratio of initiating alcohol to the lactide monomer. The resulting polyester possesses directionality with a hydroxyl terminus (from the first monomer) and a functional group at the ester terminus determined by the structure of the initiating alcohol. The latter can contain a variety of functional groups to enable attachment of a detecting means, surrogate marker, and/or payload to a nanoparticle surface.

Alternatively, functional groups can be introduced by copolymerization. Natural amino acids are sterically similar to lactic acid but offer a variety of functional groups on their side chains (—OH, —CO₂H, —NH₂, —SH, etc.). Moreover, amino acids are found in all cell types, so that the polymer degradation products are non-toxic. Monomers derived from an amino acid and lactic acid can be synthesized by standard methods and used for random copolymerization with lactide. In accordance with the present invention, nanoparticles can have functional groups on any surface to enable the attachment of an SCE-detecting means, a surrogate marker, and/or a payload. Such functional groups allow the nanostructure-based assembly to be bioengineered to accomplish specific functions, such as detect, provide notification of, and treat specific conditions, disorders, or diseases.

The detecting means of the invention can allow for applications requiring specific SCE localization or immobilization (i.e., vectoring). See Langer, R., “Tissue Engineering,” Mol Ther, 2:12-15 (2000). Detecting means including, for example, proteins, antibodies, peptides, RNA or DNA aptamers, cellular reporters or cellular ligands, can be attached to a nanoparticle surface to provide a means for vectoring the nanostructure-based assembly to a target SCE. Such SCE-detecting means may be attached covalently, including attachment via linker molecules. SCE-detecting means can also be attached to a nanoparticle surface by non-covalent linkage, for example, by absorption via hydrophobic binding or Van der Waals forces, hydrogen bonding, acid/base interactions, and electrostatic forces.

In addition, the detecting means, surrogate marker, and/or payload can be incorporated into the nanoparticle framework, which can include chitosan, PEGylated PLGA (poly(lactic-co-glycolic acid), or other PEGylated compounds. For example, a commercially available PEG-maleimide can be incorporated into chain-end thiols on the outer surface of the nanoparticles. Alternatively, the detecting means, surrogate marker, and/or payload can be incorporated into nanoparticle frameworks composed of biodegradable and/or resorbable materials including, for example, polylactide based polymers as described above.

For nanoparticles comprising a hollow void in which the surrogate marker can be contained, a surrogate marker can be loaded into the void using an electrophoretic force. (See Miller, S. A. and C. R. Martin, “Electroosmotic Flow in Carbon Nanotube Membranes,” J. Am. Chem. Soc., 123(49):12335-12342 (2001)). Alternatively, nanoparticles embedded within the synthesis membrane can be filled with a surrogate marker by vacuum filtering a solution containing the surrogate marker through the synthesis membrane. (See Parthasarathy, R. and C. R. Martin, Nature, 369:298 (1994)). For nanoparticles prepared by formation within an alumina template film prior to removal of the alumina from the underlying aluminum surface, they can be filled by simply applying a solution containing the surrogate marker to the surface of the film (where the opening to the hollow void is located) and allowing the solvent to evaporate. Multiple applications can be used, if needed.

Specific Chemical Entities (SCEs)

Many types of important antigens on cell surfaces indicate the presence of a wide variety of disease states, ranging from cancer, inflammatory disorders, and infections to cardiovascular disease. Surface cell markers can help identify a diseased cell (i.e., malignancy) in two ways: 1) by being uniquely expressed (not ordinarily present on the surface in normal cells), or 2) by being expressed in a greatly altered density (i.e., marked overexpression of a surface cell marker). For example, in the case of blood malignancies such as lymphomas and leukemias, unique markers and clusters of surface markers can be used to accurately identify blood cancers. Accordingly, SCEs of the present invention can include, without limitation, surface markers that identify disease states, including those surface markers known to identify leukemias and lymphomas via immunophenotyping.

Examples of such SCEs include, and are not limited to, (1) T cell markers (CD2, CD3, CD4, CD5, CD7, and CD8); B cell markers (CD19 and CD20); myeloid/monocytic markers (CD13, CD 14, CD15, and CD33); maturity status markers (CD34, HLA-DR, and CD10=CALLA) that form an acute leukemia surface antigen profile; (2) pan-T cell markers: CD2, CD3, CD5; CD4 (helper) and CD8 (suppressor); pan-B markers CD19 and CD20; CD5 and CD20 (co-expression frequently indicates neoplastic proliferations) that form a chronic lymphocytic leukemia (CLL) and lymphoma Profile; (3) hairy cell markers CD11c (complement receptor), CD 25 (IL-2 receptor), CD103, prolymphocytic/hairy cell marker FMC-7; B-lymphoid marker CD23 (evaluated in relationship to CD5 expression for the different diagnosis of CLL vs. MCL) that aid in diagnosing Hairy Cell Leukemia (HCL), Prolymphocytic Leukemia (PLL), or Mantle Cell Lymphoma/Leukemia; and (4) CD1, CD15, and CD30 (Ki-1) that indicate anaplastic lymphoma and Hodgkin's Disease.

Additional SCEs contemplated by the present invention include those that are located in body fluids and that are not attached to cells. Such SCEs not only include those biomarkers that are primarily released by diseased cells but also entail therapeutic and/or illicit drugs that have been imbibed.

Examples of such SCEs include, and are not limited to, the following: Alpha Fetoprotein (AFP), which is a useful tumor marker for the diagnosis and management of hepatocellular carcinoma and non-seminomatous testicular cancer; Beta2-Microglubulin (b2-M), high concentrations of which indicate active disease, cell turnover, tumor presence; the presence of inflammatory diseases (i.e., rheumatoid arthritis, systemic lupus erythematosus, Sjögren syndrome, Crohn's disease); or be a secondary indication of various lymphoproliferative diseases (leukemia, lymphoma, and multiple myeloma); Beta Human Chorionic Gonadotropin (b HCG), which is a tumor marker for gestational trophoblastic diseases, germ cell tumors of the ovary or testis, and cancers of the breast, lung, pancreas, stomach, kidney, and brain and is very helpful in assessing the efficacy of therapy in patients with testicular tumors; Carbohydrate antigen 19-9 (CA19-9), which is not organ specific but is a marker for a variety of adenocarcinomas (pancreatic, gastric, and hepatobiliary); and CA 125, which is found in most serous, endometrioid and clear cells carcinomas of the ovary.

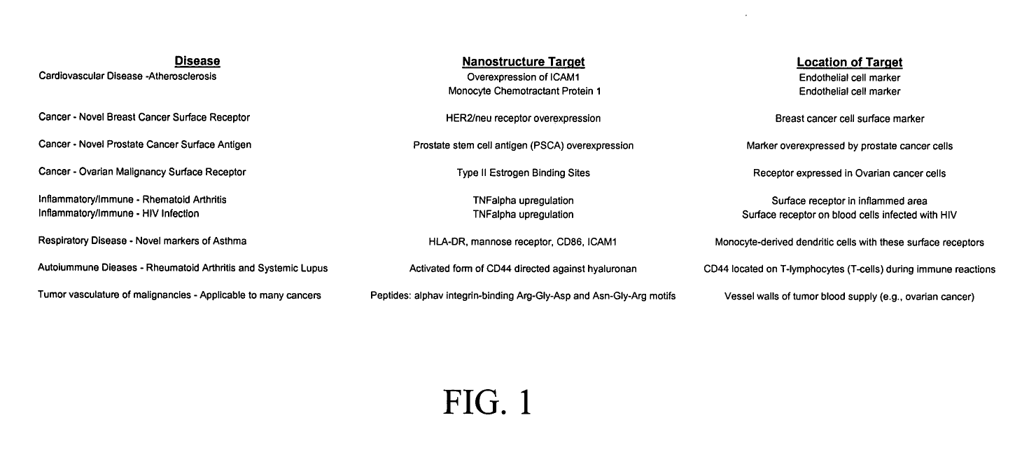

Given the arrival of new technologies such as differential screening of phage displayed libraries to identify highly novel cell surface markers specific to different types of malignancies (i.e., ovarian cancer), the utility of the nanostructure-based assemblies of the present invention to detect, notify, and monitor a wide variety of disease processes will markedly increase in the next decade. FIG. 1 illustrates certain new and older SCEs for key human maladies that can be detected using the present invention.

Means for Detecting Specific Chemical Entities (SCEs)

A nanostructure-based assembly of the invention comprises a nanoparticle, which contains a means for detecting a target SCE, a surrogate marker, and a payload. In a preferred embodiment, an SCE-detector is designed to detect a target SCE. In certain embodiments, the SCE-detector can be designed to alter the biological function of the target SCE. According to the present invention, an SCE-detector can also be designed to localize nanostructure-based assemblies within the vicinity of or into target cells for optimal release of payload (or surrogate marker).

The SCE-detector of the present invention can be an antibody specific to a target SCE. An antibody has a recognized structure that includes an immunoglobulin heavy and light chain. The heavy and light chains include an N-terminal variable region (V) and a C-terminal constant region (C). The heavy chain variable region is often referred to as “V_{H}” and the light chain variable region is referred to as “V_{L}”. The V_{H} and V_{L} chains form a binding pocket that has been referred to as F(v). See generally Davis, 3: 537, Ann. Rev. of Immunology (1985); and Fundamental Immunology 3rd Ed., W. Paul Ed. Raven Press LTD. New York (1993).

Alternatively, recombinant bispecific antibody (bsFv) molecules can be used as an SCE-detector. In a preferred embodiment, bsFv molecules that bind a T-cell protein termed “CD3” and a TAA are used as an SCE-detector in accordance with the present invention. In related embodiments, bsFv molecules are used not only to specifically bind to a target sCE but also to facilitate an immune system response. See Jost, C. R. 33: 211, Mol. Immunol (1996); Lindhofer, H. et al. 88: 465 1, Blood (1996); Chapoval, A. I. et al. 4: 571, J. of Hematotherapy (1995).

With other embodiments of the present invention, the SCE-detecting means is in the form of an aptamer.

The discovery of the SELEX™ (Systematic Evolution of Ligands by EXponential enrichment) methodology enabled the identification of aptamers that recognize molecules other than nucleic acids with high affinity and specificity (Ellington and Szostak, “In vitro selection of RNA molecules that bind specific ligands,” Nature, 346:818-822 (1990); Gold et al., “Diversity of oligonucleotide functions,” Ann. Rev. Biochem., 64:763-797 (1995); Tuerk and Gold, “Systematic evolution of ligands by exponential enrichment—RNA ligands to bacteriophage-T4 DNA-polymerase,” Science, 249:505-510 (1990)). Aptamers have been selected to recognize a broad range of targets, including small organic molecules as well as large proteins (Gold et al., supra.; Osborne and Ellington, “Nucleic acid selection and the challenge of combinatorial chemistry,” Chem. Rev., 97:349-370 (1997)).

The aptamers derived from the SELEX methodology may be utilized in the present invention. The SELEX methodology enables the production of aptamers, each of which have a unique sequence and the property of binding specifically to a desired target compound or molecule. The SELEX methodology is based on the insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets. See also Jayasena, S., “Aptamers: An Emerging Class of Molecules That Rival Antibodies for Diagnostics,” Clinical Chemistry, 45:9, 1628-1650 (1999).

Aptamers that can be used in the present invention include those described in U.S. Pat. No. 5,656,739 (hereinafter the '739 patent), which discloses the advantages of synthetic oligonucleotides as assembly templates. The '739 patent describes nucleic acids as particularly useful assembly templates because they can be selected to specifically bind nonoligonucleotide target molecules with high affinity (e.g., Tuerk and Gold (1990), supra), and because they can hybridize by complementary base pairing. Both forms of recognition can be programmably synthesized in a single molecule or hybridized into a single discrete structure.

Aptamers can be attached to proteins utilizing methods well known in the art (see Brody, E. N. and L. Gold, “Aptamers as therapeutic and diagnostic agents,” J Biotechnol, 74(1):5-13 (2000) and Brody, E. N. et al., “The use of aptamers in large arrays for molecular diagnostics,” Mol Diagn, 4(4):381-8 (1999)). For example, photo-cross-linkable aptamers allow for the covalent attachment of aptamers to proteins. Such aptamer-linked proteins can then be immobilized on a functionalized surface of a nanoparticle. For example, aptamer-linked proteins can be attached covalently to a nanoparticle end-cap or to an exterior nanoparticle surface, including attachment of the aptamer-linked protein by functionalization of the surface. Alternatively, aptamer-linked proteins can be covalently attached to a nanoparticle surface via linker molecules. Non-covalent linkage provides another method for introducing aptamer-linked proteins to a nanoparticle surface. For example, an aptamer-linked protein may be attached to an nanoparticle surface by absorption via hydrophilic binding or Van der Waals forces, hydrogen bonding, acid/base interactions, and electrostatic forces.

Payload Materials

By way of example, one embodiment of the present invention uses nanoparticle-based sensors that contain anti-oxidant genes (MnSOD, HO-1, and PON1), which are released in the presence of pro-atherogenic genes to enable treatment of atherosclerosis in a patient.

Specific payload materials include, but are not limited to, genetic material (i.e., DNA); RNA; oligonucleotides; peptides; proteins (i.e., enzymes), chemotherapeutics (anti-cancer pharmaceuticals); antibiotics; antifungal agents; anesthetics; immunomodulators (i.e., interferon, cyclosporine); anti-inflammatory and other types of pain relieving agents; autonomic drugs; cardiovascular-renal drugs; endocrine drugs; hematopoietic growth factors; blood lipid lowering drugs; AIDS drugs; modulators of smooth muscle function; antileptics; psychoactive drugs; and drugs that act on the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, metabolic systems, the immunological system, the reproductive system, the skeletal system, autacoid systems, the alimentary and excretory systems, the histamine system, and the central nervous system. Suitable agents may be selected from, for example, proteins, enzymes, hormones, polynucleotides, nucleoproteins, polysaccharides, glycoproteins, lipoproteins, polypeptides, steroids, analgesics, local anesthetics, antibiotic agents, anti-inflammatory corticosteroids, ocular drugs, and synthetic analogs of these species.

Examples of drugs which may be delivered by nanostructure-based assemblies include, but are not limited to, prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erthyrityl tetranitrate, digoxin, Intal (disodium cromoglycate), codeine, morphine, sodium salicylate, salicylic acid, meperidine hydrochloride (DEMEROL), chlophedianol. hydrochloride, epinephrine, isoproterenol, salbutamol, terbutaline, ephedrine, aminophylline, acetylcysteine, sulfanilamide, sulfadiazine, tetracycline, rifampin (rifamycin), dihydrostreptomycin, p-aminosalicylic acid, hypoglycemics tolbutamide (ORINASE), prednisone, prednisolone, prednisolone metasulfobenzoate, chlorambucil, busulfan, alkaloids, antimetabolites, 6-mercaptopurine, thioguanine, 5-fluorouracil, hydroxyurea, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erthyromycin, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, 17-α-hydroxygrogesterone acetate, 19-norprogesterone, norgestrel, norethindrone, norethisterone, norethiederone, progesterone, norgesterone, norethynodrel, aspirin, indomethacin, naproxen, fenoprofen, sulindac, indoprofen, nitroglycerin, isosorbide dinitrate, propranolol, timolol, atenolol, alprenolol, crimetidine, clonidine, imipramine, levodopa, chlorpromazine, methyldopa, dihydroxyphenylanine, theophylline, calcium gluconate, ketoprofen, ibuprofen, cephalexin, erythromycin, haloperidol, zomepirac, ferrous lactate, vincamine, phenoxybenzamine, diltiazem, milrinone, mandol, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat captopril, ramipril, famotidine, nizatidine, sucralfate, etintidine, tetratolol, minoxidil, chlordiazepoxide, diazepam, amitriptyline, and imipramine.

Further examples are proteins and peptides which include, but are not limited to, bone morphogenic proteins, insulin, colchicines, glucagons, thyroid stimulating hormone, parathyroid and pituitary hormones, calcitonin, rennin, prolactin, corticotrophin, thyrotropic hormone, follicle stimulating hormone, chorionic gonadotropin, gonadotropin releasing hormone, bovine somatotropin, porcine somatotropin, oxytocin, vasopressin, GRF, somatostatin, lypressin, pancreozymin, luteinizing hormone, LHRH, LHRH agonists and antagonists, leuprolide, interferons such as interferon alpha-2a, interferon alpha-2b, and consensus interferon, interleukins, growth hormones such as human growth hormone and its derivatives such as methione-human growth hormone and desphenylalnine human growth hormone, bovine growth hormone and porcine growth hormone, fertility inhibitors such as prostaglandins, fertility promoters, growth factors such as insulin-like growth factor, coagulation factors, human pancreas hormone releasing factor, analogs and derivatives of these compounds, and pharmaceutically acceptable salts of these compounds, or their analogs or derivatives.

Additional payload materials which can be delivered by the nanostructure-based assemblies of the invention include, but are not limited to, chemotherapeutic agents such as carboplatin, cisplatin, paclitaxel, BCNU, vincristine, camptothecin, etopside, cytokines, ribozymes, interferons, oligonucleotides and oligonucleotide sequences that inhibit translation or transcription of tumor genes, functional derivatives of the foregoing, and generally known chemotherapeutic agents such as those described in U.S. Pat. No. 5,651,986.

Surrogate Markers

As an indicator of the presence of a target SCE, the surrogate marker can be any compound that can be identified in bodily fluids including radio-labeled or fluorescent compounds, compounds that change the color of bodily fluids for detection by the naked eye, or compounds that are readily identified in bodily fluids using sensor technology.

For example, the surrogate marker can be a benzodiazepine or benzodiazepine metabolite that is detectable in urine. Benzodiazepines and their metabolites readily pass through the renal system into urine making benzodiazepines and substances with similar properties especially suitable as compliance markers. Examples of benzodiazepines or benzodiazepine metabolites that can be used in the invention include diazepam and alprazolam.

Additional surrogate markers contemplated herein include, without limitation, dimethyl sulfoxide (DMSO), acetaldehyde, acetophenone, anise, benzaldehyde, benzyl alcohol, benzyl cinnamate, cadinene, camphene, camphor, cinnamon, garlic, citronellal, cresol, cyclohexane, eucalyptol, and eugenol, eugenyl methyl ether. Such markers are particularly advantageous for use in detection in exhaled breath.

The surrogate markers of the invention also include additives that have been federally approved and categorized as GRAS (“generally recognized as safe”), which are available on a database maintained by the U.S. Food and Drug Administration Center for Food Safety and Applied Nutrition. Surrogate markers categorized as GRAS and are readily detectable in bodily fluids include, and are not limited to, sodium bisulfate, dioctyl sodium sulfosuccinate, polyglycerol polyricinoleic acid, calcium casein peptone-calcium phosphate, botanicals (i.e., chrysanthemum; licorice; jellywort, honeysuckle; lophatherum, mulberry leaf; frangipani; selfheal; sophora flower bud), ferrous bisglycinate chelate, seaweed-derived calcium, DHASCO (docosahexaenoic acid-rich single-cell oil) and ARASCO (arachidonic acid-rich single-cell oil), fructooligosaccharide, trehalose, gamma cyclodextrin, phytosterol esters, gum arabic, potassium bisulfate, stearyl alcohol, erythritol, D-tagatose, and mycoprotein.

Sensor Technology

Sensor technology is used by the present invention to detect the presence of a surrogate marker in a bodily fluid sample. The detection of a surrogate marker signifies the presence and/or quantity of a target SCE. In certain embodiments, the detection of a surrogate marker can also indicate release of payload/treatment.

The present invention contemplates using sensor technology based on surface acoustic wave (SAW) sensors. These sensors oscillate at high frequencies and respond to perturbations proportional to the mass load of certain molecules. This occurs in the vapor phase on the sensor surface. The resulting frequency shift is detected and measured by a computer. Usually, an array of sensors (4-6) is used, each coated with a different chemoselective polymer that selectively binds and/or absorbs vapors of specific classes of molecules. The resulting array, or “signature” identifies specific compounds. Sensitivity of the arrays is dependent upon the homogeneity and thickness of the polymer coating.

Surface-acoustic-wave (SAW) gas-sensors generally include a substrate with piezoelectric characteristics covered by a polymer coating, which is able to selectively absorb a surrogate marker. The variation of the resulting mass leads to a variation of its resonant frequency. This type of sensor provides very good mass-volume measures of the surrogate markers. In the SAW device, the surrogate marker is used to propagate a surface acoustic wave between sets of interdigitated electrodes. The chemoselective material is coated on the surface of the transducer. When a surrogate marker interacts with the chemoselective material coated on the substrate, the interaction results in a change in the SAW properties, such as the amplitude or velocity of the propagated wave. The detectable change in the characteristics of the wave indicates the presence and concentration of the surrogate marker (and corresponding target SCE).

A SAW vapor sensing device has been disclosed in which a layer of antibodies are attached to a surface of the SAW sensor (see Stubbs, DD et al., “Investigation of Cocaine Plumes Using Surface Acoustic Wave Immunoassay Sensors,” Anal. Chem., 75:6231-6235 (2003)). When a target antigen reacts with an antibody, the acoustic velocity is altered, causing an oscillator frequency of the SAW to shift to a different value. The subject invention contemplates usage of such SAW devices, as well as those SAW sensing devices in which aptamers (including indicator aptamers), molecular beacons, and other known SCE detectors are utilized to coat a surface of the SAW sensor.

Certain embodiments use known SAW devices described in numerous patents and publications, including U.S. Pat. Nos. 4,312,228 and 4,895,017, and Groves W. A. et al., “Analyzing organic vapors in exhaled breath using surface acoustic wave sensor array with preconcentration: Selection and characterization of the preconcentrator adsorbent,” Analytica Chimica Acta, 371:131-143 (1988).

Other types of chemical sensors known in the art that use chemoselective coating applicable to the operation of the present invention include bulk acoustic wave (BAW) devices, plate acoustic wave devices, interdigitated microelectrode (IME) devices, optical waveguide (OW) devices, electrochemical sensors, and electrically conducting sensors.

In another embodiment, the invention uses fluid sensor technology, such as commercial devices known as “artificial noses,” “electronic noses,” or “electronic tongues.” These devices are capable of qualitative and/or quantitative analysis of simple or complex gases, vapors, odors, liquids, or solutions. A number of patents and patent applications which describe fluid sensor technology include the following: U.S. Pat. Nos. 5,945,069; 5,918,257; 5,891,398; 5,830,412; 5,783,154; 5,756,879; 5,605,612; 5,252,292; 5,145,645; 5,071,770; 5,034,192; 4,938,928; and 4,992,244; and U.S. Patent Application No. 2001/0050228. Certain sensitive, commercial off-the-shelf electronic noses, such as those provided by Cyrano Sciences, Inc. (“CSI”) (i.e., CSI's portable Electronic Nose and CSI's Nose-Chip™ integrated circuit for odor-sensing—U.S. Pat. No. 5,945,069), can be used in the present invention to detect the presence of detectable markers in bodily fluid samples.

Other embodiments of the present invention use sensor technology selected from semiconductive gas sensors; mass spectrometers; and IR, UV, visible, or fluorescence spectrophotometers. With these sensors, a surrogate marker changes the electrical properties of the semiconductors by making their electrical resistance vary, and the measurement of these alternatives allows the determination of the concentration of detectable markers present in the sample. The methods and apparatus used for detecting surrogate markers generally have a brief detection time of a few seconds.

Additional recent sensor technologies included in the present invention include apparatus having conductive-polymer gas-sensors (“polymeric”), aptamer biosensors, and amplifying fluorescent polymer (AFP) sensors.

Conductive-polymer gas-sensors (also referred to as “chemoresistors”) are coated with a film sensitive to the molecules of certain detectable markers. On contact with the molecules, the electric resistance of the sensors change and the measurement of the variation of this resistance enable the concentration of the detected substance (i.e., surrogate marker and corresponding target SCE) to be determined. An advantage of this type of sensor is that it functions at temperatures close to ambient. Different sensitivities for detecting different detectable markers can be obtained by modifying or choosing an alternate conductive polymer.

Polymeric gas sensors can be built into an array of sensors, where each sensor responds to different gases and augment the selectivity of the surrogate marker.

Aptamer biosensors can be utilized in the present invention for detecting the presence of detectable surrogate markers in bodily fluid samples. Aptamer biosensors are resonant oscillating quartz sensors that can detect minute changes in resonance frequencies due to modulations of mass of the oscillating system, which results from a binding or dissociation event.

Similarly, amplifying fluorescent polymer (AFP) sensors may be utilized in the present invention for detecting the presence of detectable surrogate markers in bodily fluid samples. AFP sensors are extremely sensitive and highly selective chemosensors that use amplifying fluorescent polymers. When vapors bind to thin films of the polymers, the fluorescence of the film decreases. A single molecule binding event quenches the fluorescence of many polymer repeat units, resulting in an amplification of the quenching. The binding of surrogate markers to the film is reversible, therefore the films can be reused.

In accordance with the present invention, competitive binding immunoassays can be used to test a bodily fluid sample for the presence of surrogate markers. Immunoassay tests generally include an absorbent, fibrous strip having one or more reagents incorporated at specific zones on the strip. The bodily fluid sample is deposited on the strip and by capillary action the sample will migrate along the strip, entering specific reagent zones in which a chemical reaction may take place. At least one reagent is included which manifests a detectable response, for example a color change, in the presence of a minimal amount of a surrogate marker of interest. Patents that describe immunoassay technology include the following: U.S. Pat. Nos. 5,262,333 and 5,573,955.

Other embodiments of the present invention use flow cytometers to analyze bodily fluid samples for surrogate markers. Flow cytometry is a technique that is used to determine certain physical and chemical properties of microscopic biological particles by sensing certain optical properties of the particles. To do so, the particles are arranged in single file using hydrodynamic focusing within a sheath fluid. The particles are then individually interrogated by a light beam. Each particle scatters the light beam and produces a scatter profile. The scatter profile is often identified by measuring the light intensity at different scatter angles. Certain physical and/or chemical properties of each particle can then be determined from the scatter profile. Patents that describe flow cytometry technology include the following: U.S. Pat. Nos. 6,597,438; 6,097,485; 6,007,775; and 5,716,852.

Compositions containing nanostructure-based assemblies in accordance with the present invention can be administered utilizing methods known to the skilled artisan. In one aspect of the invention, the compositions are formulated in admixture with a pharmaceutically acceptable carrier and optionally, with other therapeutic and/or prophylactic ingredients.

In general, it is preferable to administer a pharmaceutical composition of the invention in orally or nasally (i.e., inhalation) administrable form, but formulations may be administered via parenteral, intravenous, intramuscular, transdermal (i.e., topical), buccal, subcutaneous, transmucosal, suppository or other route. Intravenous and intramuscular compositions are preferably administered in sterile saline. One of ordinary skill in the art may modify the compositions of the invention within the teachings of the specification to provide numerous formulations for a particular route of administration without rendering the compositions of the present invention unstable or compromising its therapeutic activity. In particular, a modification of a desired compound to render it more soluble in water or other vehicle, for example, may be easily accomplished by routine modification (salt formulation, esterification).

According to the present invention, compositions can be delivered to the patient parenterally (i.e., intravenously, intramuscularly). For such forms of administration, the compositions can be formulated into solutions or suspensions, or in lyophilized forms for conversion into solutions or suspensions before use. Sterile water, physiological saline (i.e., phosphate buffered saline (PBS)) can be used conveniently as the pharmaceutically acceptable carriers or diluents. Conventional solvents, surfactants, stabilizers, pH balancing buffers, anti-bacterial agents, chelating agents, and antioxidants can all be used in the these formulations, including but not limited to acetates, citrates or phosphates buffers, sodium chloride, dextrose, fixed oils, glycerine, polyethylene glycol, propylene glycol, benzyl alcohol, methyl parabens, ascorbic acid, sodium bisulfite, and the like. These formulation can be stored in any conventional containers such as vials, ampoules, and syringes.

Sterile injectable solutions of the compositions of the invention can be prepared by incorporating the nanostructure-based assemblies in required amounts in an appropriate solvent with one or a combination of ingredients as required, followed by sterilization. Generally, dispersions are prepared by incorporating the nanostructure-based assemblies into a sterile vehicle that contains a basic dispersion medium, and the other required ingredients. Preparation of sterile powders for sterile injectable solutions include vacuum drying and freeze-drying that yield a powder containing the active ingredient and any desired ingredients to form a sterile solution.

The compositions of the invention can also be delivered orally in enclosed gelatin capsules or compressed tablets. Capsules and tablets can be prepared in any conventional techniques. For example, the active compounds can be incorporated into a formulation, which includes pharmaceutically acceptable carriers such as excipients (i.e., starch, lactose), binders (i.e., gelatin, cellulose, gum tragacanth), disintegrating agents (i.e., alginate, Primogel, and corn starch), lubricants (i.e., magnesium stearate, silicon dioxide), and sweetening or flavoring agents (i.e., glucose, sucrose, saccharin, methyl salicylate, and peppermint). Various coatings can also be prepared for the capsules and tablets to modify the flavors, tastes, colors, and shapes of the capsules and tablets. In addition, liquid carriers such as fatty oil can also be included in capsules

The nanostructure-based assemblies of the invention can be added to a medical formulation by homogeneously mixing them throughout the formulation or solution of the therapeutic medication. Alternatively, the nanostructure-based assemblies are formed as a film or coating on a tablet or capsule containing the therapeutic medication. If more than one medication has been prescribed, a separate first and/or second detectable marker can be used in association with each medication. Preferably the first and/or second markers of the invention have biological half-lives of between 24 and 48 hours so that they will appear in a sample of bodily fluids taken from the patient.

### EXAMPLE 1

### Systems and Methods for Testing Heroin Use

In one embodiment, a patient suffering from heroin addiction is administered a composition comprising nanoparticle-based assemblies of the invention. The nanoparticle-based assemblies are designed to detect the drug heroin. In one embodiment, the nanoparticle-based assemblies contain a nanoparticle, a surrogate marker, and an SCE-detector. Preferably, the SCE-detector is an aptamer that is designed to be specific for heroin (heroin-aptamer). The heroin-aptamer and the surrogate marker (heroin-surrogate marker) are attached to a surface of the nanoparticle.

In a preferred embodiment, the heroin-aptamer is attached to an end-cap of a hollow nanoparticle that contains therein the heroin-surrogate marker. The heroin-aptamer is designed so that upon interaction with heroin, the end-cap is released from the nanoparticle to release the heroin-surrogate marker. The heroin-surrogate marker is readily detectable in bodily fluid samples taken from the patient.

To test for heroin use, the nanoparticle-based assemblies are administered to the patient and then a sample of the patient's bodily fluid (i.e., urine, breath, blood) is acquired. Where heroin is present in the patient, the heroin interacts with the heroin-aptamer and “uncaps” the nanoparticle, thus releasing the heroin-surrogate marker for identification in the bodily fluid sample. Any one of a number of previously disclosed sensor technologies is then used to detect the heroin-surrogate marker, where the heroin-surrogate marker indicates presence of heroin in the patient's body.

### EXAMPLE 2

### Treatment of Atherosclerosis

In another embodiment of the invention, a patient suffering from atherosclerosis is administered a composition comprising nanoparticle-based assemblies to diagnose and treat atherosclerosis. The nanoparticle-based assembly comprises a nanoparticle; a surrogate marker; a payload; and an SCE-detector. Treatment of atherosclerosis (payload) comprises anti-oxidant genes (MnSOD, HO-1 and PON1) that utilize the patient's own hormonal changes to offset atherosclerotic disease progression. The SCE-detector is designed to detect biomarkers of atherosclerosis (i.e., ICAM-1, VCAM-1, or LOX-1). ICAM-1, VCAM-1, and LOX-1 are pro-atherogenic genes in human coronary endothelial cells that are regulated by cytokine levels (IL1, TNF, IL-6).

Once the SCE-detector is in the presence of an atherosclerosis biomarker, it causes the release of the anti-oxidant genes and the surrogate marker. The antioxidant genes not only alter the development of atherosclerosis but also afford cytoprotective treatment to vascular endothelium to prevent the development of atherosclerosis. The surrogate marker is an indicator in bodily fluid samples that pro-atherogenic biomarkers are present in the patient as well as an indicator that antioxidant genes have been administered to the patient.

### EXAMPLE 3

### Diagnosis and Treatment of Glycogen Storage Disorder

Glycogen is readily detectable in bodily fluids (i.e., blood) using a nanoparticle-based assembly of the invention. According to the present invention, the nanoparticle-based assembly comprises a nanoparticle, a surrogate marker, and an SCE-detector that is designed to bind to the glycogen and to act upon the glycogen in a fashion similar to muscle phosphorylase to safely break down glycogen. Binding of the SCE-detector to glycogen causes the release of the surrogate marker for detection. Thus, with the present invention, it is possible to not only diagnose a specific disease/condition in a patient but also to treat it and ensure patient compliance with the treatment regimen. In addition, the method of the present invention can evaluate pharmacodynamics and pharmacokinetics for drug interventions in individuals.

### EXAMPLE 4

### Assessment of Blood and Diagnosis/Treatment of Blood-Based Diseases

In one embodiment, the nanostructure-based assemblies of the invention can be used to differentiate and signal types of blood cells and their concentrations in the patient. For example, levels of red blood cells (RBCs), white blood cells (WBCs), and platelets can be assessed using the systems and methods of the invention to diagnose and/or treat hematopoiesis abnormalities such as leukemia or assess changes in cellular contect (e.g., RBC content).

Accordingly, the subject invention is useful in diagnosing and/or treating blood-based diseases or disorders including, without limitation, hemorrhagic diathesis (i.e., hemophilia, von Willebrand disease, Alexander's disease, Telfer's disease, Owren's parahemophilia, prothrombin deficiency); non-hemorrhagiparous coagulopathies (i.e., Fletcher factor deficiency, Flaujeac factor deficiency); thrombophilic coagulopathies (i.e., Ratnoff's disease, thrombomodulin deficiency); thrombocytopenia; anemias; and alterations in white blood cells (i.e., Pelger-Huët anomaly (PHA); Chediak-Higashi syndrome (CHS); Hegglin-May anomaly (HMA)).

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application

1. A method for diagnosis of a condition, disease, or disorder, comprising:
(a) administering to a patient a composition comprising at least one nanoparticle-based assembly, wherein the nanoparticle-based assembly comprises a nanoparticle; a surrogate marker, and a means for detecting a specific chemical entity (SCE);
(b) obtaining a sample of bodily fluid from the patient;
(c) applying sensor technology to the sample of bodily fluid to detect the presence of the surrogate marker.

2. The method according to claim 1, wherein the nanoparticle is a nanotube.

3. The method according to claim 1, wherein SCE-detecting means is selected from the group consisting of an antibody, a protein, and an aptamer.

4. The method according to claim 1, wherein the surrogate marker is selected from the group consisting of DMSO, benzodiazepine, a benzodiazepine metabolite, acetaldehyde, acetophenone, anise, benzaldehyde, benzyl alcohol, benzyl cinnamate, cadinene, camphene, camphor, cinnamon, citronellal, cresol, cyclohexane, eucalyptol, and eugenol, eugenyl methyl ether.

5. The method according to claim 1, wherein the surrogate marker is selected from the group consisting of sodium bisulfate, dioctyl sodium sulfosuccinate, polyglycerol polyricinoleic acid, calcium casein peptone-calcium phosphate, botanicals (i.e., chrysanthemum; licorice; jellywort, honeysuckle; lophatherum, mulberry leaf; frangipani; selfheal; sophora flower bud), ferrous bisglycinate chelate, seaweed-derived calcium, DHASCO (docosahexaenoic acid-rich single-cell oil) and ARASCO (arachidonic acid-rich single-cell oil), fructooligosaccharide, trehalose, gamma cyclodextrin, phytosterol esters, gum arabic, potassium bisulfate, stearyl alcohol, erythritol, D-tagatose, and mycoprotein.

6. The method according to claim 1, wherein the bodily fluid sample is selected from the group consisting of exhaled breath, whole blood, blood plasma, urine, semen, saliva, lymph fluid, meningal fluid, amniotic fluid, glandular fluid, sputum, feces, sweat, mucous, and cerebrospinal fluid.

7. The method according to claim 1, wherein the bodily fluid sample is a separated fraction of a solution or mixture containing homogenized solid materials selected from the group consisting of feces, tissues, and biopsy samples.

8. The method according to claim 1, wherein the SCE-detecting means has a specific action on compounds selected from the group consisting of acetaldehyde, acetone, ammonia, carbon monoxide, chloroform, diethylamine, hydrogen, isoprene, methanethiol, methylethylketone, O-toluidine, pentane sulfides and sulfides, H₂S, MeS, Me₂S, αII-spectrin breakdown products and/or isoprostanes, prostate specific antigen, and GLXA.

9. The method according to claim 1, wherein the SCE-detecting means has a specific action on compounds selected from the group consisting of illicit, illegal, or controlled substances; allergens; toxins; carcinogens; infectious agents; and cell markers for diseases.

10. The method according to claim 1, wherein the SCE-detecting means has a specific action on compounds selected from the group consisting of amphetamines, analgesics, barbiturates, club drugs, cocaine, crack cocaine, depressants, designer drugs, ecstasy, Gamma Hydroxy Butyrate, hallucinogens, heroin, morphine, inhalants, ketamine, lysergic acid diethylamide, marijuana, methamphetamines, opiates, narcotics, phencyclidine, prescription drugs, psychedelics, Rohypnol, steroids, stimulants, pollen, spores, dander, peanuts, eggs, shellfish, mercury, lead, other heavy metals, Clostridium Difficile toxin, acetaldehyde, beryllium compounds, chromium, dichlorodiphenyltrichloroethane (DDT), estrogens, N-methyl-N′-nitro-N-nitrosoguanidine (MNNG), radon, Bordettella bronchiseptica, citrobacter, Escherichia coli, hepatitis viruses, herpes, immunodeficiency viruses, influenza virus, Listeria, micrococcus, mycobacterium, rabies virus, rhinovirus, rubella virus, Salmonella, yellow fever virus, T cell markers, B cell markers, myeloid/monocytic markers, maturity status markers, α-Fetoprotein, β2-Microglobulin, and Beta Human Chorionic Gonadotropin (b HCG.

11. The method according to claim 1, wherein the nanoparticle is formed with an interior void that contains the surrogate marker, wherein the nanoparticle has at least one open end to provide access to the interior void.

12. The method according to claim 11, wherein the interior void also contains a payload.

13. The method according to claim 11, wherein the nanoparticles further includes an end-cap to block the open end.

14. The method according to claim 13, wherein the end-cap is a particle that has a maximum dimension of less than 100 μm.

15. The method according to claim 13, wherein the end-cap is attached to the nanoparticle by covalent bonds.

16. The method according to claim 13, wherein the nanoparticle is in the form of a tubular body; and wherein the SCE-detecting means is attached to the end-cap.

17. The method according to claim 1, wherein the nanoparticle is composed of silica.

18. The method according to claim 1, wherein the nanoparticle is composed of a polymer.

19. The method according to claim 18, wherein the SCE-detecting means is attached to a surface of the nanoparticle using copolymerization.

20. The method according to claim 18, wherein the polymer nanoparticle is composed of polymers selected from the group consisting of polystyrene, polyorganosiloxane, poly(methyl methacrylate), polystyrene, polylactic acids, and other biodegradable polymers, acrylic latexes, polyorganosiloxane, cellulose, polyethylene, poly(vinyl chloride), poly(ethyl methacrylate), poly(tetrafluoroethylene), poly(4-iodostyrene/divinylbenzene), poly(4-vinylpyridine/divinylbenzene), poly(styrene/divinyl benzene), crosslinked melamine particles, phenolic polymer colloids, polyamide 6/6, natural rubber, and naturally occurring biopolymers.

21. The method according to claim 18, wherein the polymer nanoparticle is composed of biodegradable polymers selected from the group consisting of poly(caprolactone), poly(glycolic acid), poly(lactic acid), poly(hydroxybutryate), poly(adipic anhydride), poly(maleic anhydride), polydioxanone, polyamines, polyamides, polyurethanes, polyesteramides, polyorthoesters, polyacetals, polyketals, polycarbonates, polyorthocarbonates, polyphosphazenes, poly(malic acid), poly(amino acids), polyvinylpyrrolidone, poly(methyl vinyl ether), poly(alkylene oxalate), poly(alkylene succinate), polyhydroxycellulose, chitin, chitosan, and copolymers.

22. The method according to claim 18, wherein the polymer nanoparticle is composed of biocompatible polymers selected from the group consisting of poly(lactide-co-glycolide), poly(ethylene glycol), and copolymers of poly(ethylene oxide) with poly(L-Lactic acid) or with poly(3-benzyl-L-aspartate.

23. The method according to claim 1, wherein the SCE-detecting means is incorporated into the nanoparticle.

24. The method according to claim 1, wherein the nanoparticle is produced in a shape selected from a group consisting of spherical; elliptical; cubic; cylindrical; tetrahedron; polyhedral; irregular-prismatic; icosahedral; and cubo-octahedral.

25. The method according to claim 1, wherein the nanoparticle has a dimension less than 500 nm.

26. The method according to claim 1, wherein the surface of the nanoparticle is stealthy.

27. A method for diagnosis and treatment of a condition, disease, or disorder, comprising:
(a) administering to a patient a composition comprising at least one nanoparticle-based assembly, wherein the nanoparticle-based assembly comprises a nanoparticle; a surrogate marker, a means for detecting a specific chemical entity (SCE), and a payload;
(b) obtaining a sample of bodily fluid from the patient;
(c) applying sensor technology to the sample of bodily fluid to detect the presence of the surrogate marker.

28. The method according to claim 27, wherein the nanoparticle is a nanotube.

29. The method according to claim 27, wherein SCE-detecting means is selected from the group consisting of an antibody, a protein, and an aptamer.

30. The method according to claim 27, wherein the surrogate marker is selected from the group consisting of benzodiazepine, a benzodiazepine metabolite, acetaldehyde, DMSO, acetophenone, anise, benzaldehyde, benzyl alcohol, benzyl cinnamate, cadinene, camphene, camphor, cinnamon, citronellal, cresol, cyclohexane, eucalyptol, and eugenol, eugenyl methyl ether.

31. The method according to claim 27, wherein the surrogate marker is selected from the group consisting of sodium bisulfate, dioctyl sodium sulfosuccinate, polyglycerol polyricinoleic acid, calcium casein peptone-calcium phosphate, botanicals (i.e., chrysanthemum; licorice; jellywort, honeysuckle; lophatherum, mulberry leaf; frangipani; selfheal; sophora flower bud), ferrous bisglycinate chelate, seaweed-derived calcium, DHASCO (docosahexaenoic acid-rich single-cell oil) and ARASCO (arachidonic acid-rich single-cell oil), fructooligosaccharide, trehalose, gamma cyclodextrin, phytosterol esters, gum arabic, potassium bisulfate, stearyl alcohol, erythritol, D-tagatose, and mycoprotein.

32. The method according to claim 27, wherein the bodily fluid sample is selected from the group consisting of exhaled breath, whole blood, blood plasma, urine, semen, saliva, lymph fluid, meningal fluid, amniotic fluid, glandular fluid, sputum, feces, sweat, mucous, and cerebrospinal fluid.

33. The method according to claim 27, wherein the bodily fluid sample is a separated fraction of a solution or mixture containing homogenized solid materials selected from the group consisting of feces, tissues, and biopsy samples.

34. The method according to claim 27, wherein the SCE-detecting means has a specific action on compounds selected from the group consisting of acetaldehyde, acetone, ammonia, carbon monoxide, chloroform, diethylamine, hydrogen, isoprene, methanethiol, methylethylketone, O-toluidine, pentane sulfides and sulfides, H₂S, MeS, Me₂S, αII-spectrin breakdown products and/or isoprostanes, prostate specific antigen, and GLXA.

35. The method according to claim 27, wherein the SCE-detecting means has a specific action on compounds selected from the group consisting of illicit, illegal, or controlled substances; allergens; toxins; carcinogens; infectious agents; and cell markers for diseases.

36. The method according to claim 27, wherein the SCE-detecting means has a specific action on compounds selected from the group consisting of amphetamines, analgesics, barbiturates, club drugs, cocaine, crack cocaine, depressants, designer drugs, ecstasy, Gamma Hydroxy Butyrate, hallucinogens, heroin, morphine, inhalants, ketamine, lysergic acid diethylamide, marijuana, methamphetamines, opiates, narcotics, phencyclidine, prescription drugs, psychedelics, Rohypnol, steroids, stimulants, pollen, spores, dander, peanuts, eggs, shellfish, mercury, lead, other heavy metals, Clostridium Difficile toxin, acetaldehyde, beryllium compounds, chromium, dichlorodiphenyltrichloroethane (DDT), estrogens, N-methyl-N′-nitro-N-nitrosoguanidine (MNNG), radon, Bordettella bronchiseptica, citrobacter, Escherichia coli, hepatitis viruses, herpes, immunodeficiency viruses, influenza virus, Listeria, micrococcus, mycobacterium, rabies virus, rhinovirus, rubella virus, Salmonella, yellow fever virus, T cell markers, B cell markers, myeloid/monocytic markers, maturity status markers, α-Fetoprotein, β2-Microglobulin, and Beta Human Chorionic Gonadotropin (b HCG.

37. The method according to claim 27, wherein the nanoparticle is formed with an interior void that contains the surrogate marker, wherein the nanoparticle has at least one open end to provide access to the interior void.

38. The method according to claim 37, wherein the interior void also contains a payload.

39. The method according to claim 37, wherein the nanoparticles further includes an end-cap to block the open end.

40. The method according to claim 39, wherein the end-cap is a particle that has a maximum dimension of less than 100 μm.

41. The method according to claim 39, wherein the end-cap is attached to the nanoparticle by covalent bonds.

42. The method according to claim 39, wherein the nanoparticle is in the form of a tubular body; and wherein the SCE-detecting means is attached to the end-cap.

43. The method according to claim 27, wherein the nanoparticle is composed of silica.

44. The method according to claim 27, wherein the nanoparticle is composed of a polymer.

45. The method according to claim 44, wherein the SCE-detecting means is attached to a surface of the nanoparticle using copolymerization.

46. The method according to claim 44, wherein the polymer nanoparticle is composed of polymers selected from the group consisting of polystyrene, polyorganosiloxane, poly(methyl methacrylate), polystyrene, polylactic acids, and other biodegradable polymers, acrylic latexes, polyorganosiloxane, cellulose, polyethylene, poly(vinyl chloride), poly(ethyl methacrylate), poly(tetrafluoroethylene), poly(4-iodostyrene/divinylbenzene), poly(4-vinylpyridine/divinylbenzene), poly(styrene/divinyl benzene), crosslinked melamine particles, phenolic polymer colloids, polyamide 6/6, natural rubber, and naturally occurring biopolymers.

47. The method according to claim 44, wherein the polymer nanoparticle is composed of biodegradable polymers selected from the group consisting of poly(caprolactone), poly(glycolic acid), poly(lactic acid), poly(hydroxybutryate), poly(adipic anhydride), poly(maleic anhydride), polydioxanone, polyamines, polyamides, polyurethanes, polyesteramides, polyorthoesters, polyacetals, polyketals, polycarbonates, polyorthocarbonates, polyphosphazenes, poly(malic acid), poly(amino acids), polyvinylpyrrolidone, poly(methyl vinyl ether), poly(alkylene oxalate), poly(alkylene succinate), polyhydroxycellulose, chitin, chitosan, and copolymers.

48. The method according to claim 44, wherein the polymer nanoparticle is composed of biocompatible polymers selected from the group consisting of poly(lactide-co-glycolide), poly(ethylene glycol), and copolymers of poly(ethylene oxide) with poly(L-Lactic acid) or with poly(β-benzyl-L-aspartate.

49. The method according to claim 27, wherein the SCE-detecting means is incorporated into the nanoparticle.

50. The method according to claim 27, wherein the nanoparticle is produced in a shape selected from a group consisting of spherical; elliptical; cubic; cylindrical; tetrahedron; polyhedral; irregular-prismatic; icosahedral; and cubo-octahedral.

51. The method according to claim 27, wherein the nanoparticle has a dimension less than 500 nm.

52. The method according to claim 27, wherein the surface of the nanoparticle is stealthy.

53. The method according to claim 27, wherein the payload is selected from the group consisting of genetic materials; RNA; oligonucleotides; polynucleotides; peptides; proteins; enzymes; hormones; steroids; chemotherapeutics; antibiotics; antifungal agents; anesthetics; immunomodulators; anti-inflammatory agents; pain relieving agents; autonomic drugs; cardiovascular-renal drugs; endocrine drugs; hematopoietic growth factors; blood lipid lowering drugs; AIDS drugs; modulators of smooth muscle function; antileptics; psychoactive drugs; and drugs that act on the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, metabolic systems, the immunological system, the reproductive system, the skeletal system, autacoid systems, the alimentary and excretory systems, the histamine system, and the central nervous system.

54. The method according to claim 53, wherein the payload is selected from the group consisting of prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erthyrityl tetranitrate, digoxin, Intal (disodium cromoglycate), codeine, morphine, sodium salicylate, salicylic acid, meperidine hydrochloride (DEMEROL), chlophedianol hydrochloride, epinephrine, isoproterenol, salbutamol, terbutaline, ephedrine, aminophylline, acetylcysteine, sulfanilamide, sulfadiazine, tetracycline, rifampin (rifamycin), dihydrostreptomycin, p-aminosalicylic acid, hypoglycemics tolbutamide (ORINASE), prednisone, prednisolone, prednisolone metasulfobenzoate, chlorambucil, busulfan, alkaloids, antimetabolites, 6-mercaptopurine, thioguanine, 5-fluorouracil, hydroxyurea, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erthyromycin, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, 17-α-hydroxygrogesterone acetate, 19-norprogesterone, norgestrel, norethindrone, norethisterone, norethiederone, progesterone, norgesterone, norethynodrel, aspirin, indomethacin, naproxen, fenoprofen, sulindac, indoprofen, nitroglycerin, isosorbide dinitrate, propranolol, timolol, atenolol, alprenolol, crimetidine, clonidine, imipramine, levodopa, chlorpromazine, methyldopa, dihydroxyphenylanine, theophylline, calcium gluconate, ketoprofen, ibuprofen, cephalexin, erythromycin, haloperidol, zomepirac, ferrous lactate, vincamine, phenoxybenzamine, diltiazem, milrinone, mandol, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat captopril, ramipril, famotidine, nizatidine, sucralfate, etintidine, tetratolol, minoxidil, chlordiazepoxide, diazepam, amitriptyline, and imipramine.

55. The method according to claim 53, wherein the payload is selected from the group consisting of bone morphogenic proteins, insulin, colchicines, glucagons, thyroid stimulating hormone, parathyroid hormones, pituitary hormones, calcitonin, rennin, prolactin, corticotrophin, thyrotropic hormone, follicle stimulating hormone, chorionic gonadotropin, gonadotropin releasing hormone, bovine somatotropin, porcine somatotropin, oxytocin, vasopressin, GRF, somatostatin, lypressin, pancreozymin, luteinizing hormone, LHRH, LHRH agonists and antagonists, leuprolide, interferons, consensus interferon, interleukins, growth hormones, bovine growth hormone, porcine growth hormone, fertility inhibitors, fertility promoters, growth factors, coagulation factors, and human pancreas hormone releasing factor.

56. The method according to claim 53, wherein the payload is a chemotherapeutic selected from the group consisting of carboplatin, cisplatin, paclitaxel, BCNU, vincrtistine, camptothecin, etopside, cytokines, ribozymes, interferons, oligonucleotides, and oligonucleotides that inhibit translation or transcription of tumor genes.
